(19) United States
(12) Patent Application Publication
(10) Pub. No.: US 2026/0117276 A1
(43) Pub. Date: Apr. 30, 2026

(54) Frozen Biofluids Mitochondrial Bioenergetics Function Assessment Method for Traumatic Brain Injury and Polytrauma

(71) Applicant: The Government of the United States, as Represented by the Director of the Defense Health Agency, Fort Detrick, MD (US)

(72) Inventors: Jignesh D. Pandya, Germantown, MD (US); Deborah A. Shear, Columbia, MD (US); Hiren Modi, Germantown, MD (US); Sudeep Musyaju, Olney, MD (US); Anke H. Scultetus, Chester, MD (US)

(73) Assignee: The Government of the United States, as Represented by the Director of the Defense Health Agency, Fort Detrick, MD (US)

(21) Appl. No.: 19/369,749

(22) Filed: Oct. 27, 2025

Related U.S. Application Data
(60) Provisional application No. 63/712,571, filed on Oct. 28, 2024.

(51) Int. Cl. C12Q 1/25; C12Q 1/26; G01N 1/28

(52) U.S. Cl. CPC C12Q 1/25; C12Q 1/26; G01N 1/28; G01N 2333/90206; G01N 2333/90209; G01N 2333/90216

(57) ABSTRACT

A method of assessing mitochondria bioenergetics includes obtaining a frozen tissue or frozen biofluid sample of an individual and measuring mitochondria bioenergetics of the frozen tissue or frozen biofluid sample using a substrate for the frozen tissue or biofluid sample. The substrate may include at least one of Glucose; Alamethicin, Nicotinamide adenine dinucleotide (H), and Cytochrome C; Succinate and Rotenone; or Ascorbate+TMPD (Tetramethyl phenylenediamine).

This application claims priority to U.S. Ser. No. 63/712,571 filed on Oct. 28, 2024, the entirety of which is incorporated herein by reference.

### FIELD OF INVENTION

The present invention is directed to identification and characterization of mitochondrial bioenergetics in a frozen tissue or a frozen biofluid sample. The analysis of mitochondrial bioenergetics may serve as qualitative and/or quantitative biomarkers for diagnosing or validating a health condition or for evaluating treatment efficacy following at least one of traumatic brain injury (TBI), polytrauma, or other Central Nervous System (CNS) injury associated neurodegenerative diseases.

### BACKGROUND OF INVENTION

Mitochondrial oxidative function is an essential parameter to understand cellular energy metabolism in health and disease conditions. Cellular energy response through an oxidative phosphorylation (e.g., OXPHOS or ATP synthesis) assessment method is considered to be the gold standard assay for the measurement of mitochondrial oxidative function, as it reflects the activity of the electron transport chain (ETC) enzyme complexes working together with Complex V (ATP synthase).

A major limitation of mitochondrial bioenergetics function analysis is that it requires fresh tissue or isolated mitochondrial samples to conduct assessments. This problem is a barrier to translate basic research into clinical practice since samples cannot be available to perform respiratory assessments. There is a clear need to develop and refine a respiratory assessment method to evaluate mitochondrial functions, for example, from human biofluid and tissue biobanks.

There is unmet need of establishing reliable methods for evaluating frozen biofluids (e.g., buffy coat, platelets or platelets rich plasma) mitochondrial functions following neurotrauma and refinement of biofluid sample collections without compromising their respiratory function during isolation procedure.

There remains a need to assess mitochondrial bioenergetics in frozen tissue or frozen biofluid samples for assessing mitochondrial bioenergetics function in various human diseases or health conditions.

### SUMMARY OF INVENTION

The present invention provides in a first embodiment a method of assessing mitochondria bioenergetics comprising obtaining a frozen tissue or frozen biofluid sample of an individual, and measuring mitochondria bioenergetics of the frozen tissue or frozen biofluid sample.

The present invention provides in a second embodiment further to any of the previous embodiments a method wherein the measuring comprises measuring mitochondrial oxidative function by respirometry function. In embodiments, Complex I, II and IV ETC chain activity assessments may be made using identical biofluid samples together under similar experimental conditions.

The present invention provides in a third embodiment further to any of the previous embodiments a method wherein the measuring comprises measuring mitochondrial ETC Complex I, II, and IV activities in frozen biofluids obtained after penetrating TBI or Controlled Cortical Impact (CCI) injury of TBI or pre- or post-injury.

The present invention provides in a fourth embodiment further to any of the previous embodiments a method wherein the measuring comprises using a substrate for the frozen sample. The substrate may be at least one of Glucose, Alamethicin, Nicotinamide adenine dinucleotide (H), and Cytochrome C; Succinate and Rotenone; or Ascorbate+TMPD (Tetramethyl phenylenediamine).

The present invention provides in a fifth embodiment further to any of the previous embodiments a method wherein the measuring comprises measuring at least one of the ETC enzyme complexes.

The present invention provides in a sixth embodiment further to any of the previous embodiments a method wherein the mitochondrial bioenergetics comprises measuring at least one of ETC enzymes i.e., Complex I, Complex II, or Complex IV.

The present invention provides in a seventh embodiment further to any of the previous embodiments a method wherein the frozen tissue or frozen biofluid samples were preserved after collection at intervals ranging from 30 minutes to 14 days post-TBI.

The present invention provides in an eighth embodiment further to any of the previous embodiments a method wherein a frozen biofluid sample comprises blood platelets.

The present invention provides in a ninth embodiment further to any of the previous embodiments a method wherein a frozen biofluid sample comprises buffy coat.

The present invention provides in a tenth embodiment further to any of the previous embodiments a method wherein a frozen biofluid sample comprises blood plasma.

The present invention provides in an eleventh embodiment further to any of the previous embodiments a method wherein the frozen tissue or frozen biofluid sample is from an individual who has traumatic brain injury.

The present invention provides in a twelfth embodiment further to any of the previous embodiments a method further comprising identifying a health status or condition of the individual based on the measuring.

The present invention provides in a thirteenth embodiment further to any of the previous embodiments a method further comprising diagnosing moderate to severe traumatic brain injury or polytrauma based on said measuring.

### BRIEF DESCRIPTION OF THE DRAWINGS

FIG. 1A illustrates a workflow diagram of blood processing to isolate the different cell types (e.g., buffy coat, platelets or platelets rich plasma) to measure the mitochondrial respiration under different conditions. FIG. 1B is a table that shows different sets (combination) of traditional substrates and substrates used according to the present invention to measure individual ETC Complexes activity under fresh and frozen tissue and mitochondrial isolation conditions.

FIGS. 2A-2B show brain mitochondrial respiration measurement at 24 h post-injury in rat samples from Sham and penetrating TBI (PTBI) conditions. FIG. 2A shows measured mitochondrial respiration (Complex I and Complex II) using traditional substrates. FIG. 2B shows measured ETC Complex I, II and Complex IV enzyme activity using substrates according to the present invention.

FIGS. 3A-3D are graphs that represent biofluids (platelets and buffy coat) mitochondrial respiration measurements at 24 h post-injury in rat samples from Sham and following PTBI. FIGS. 3A-3B show platelet samples mitochondrial respiration using traditional substrates and substrates according to the present invention at 24 hours post-injury PTBI samples. FIGS. 3C-3D show buffy coat samples mitochondrial respiration using traditional substrates and substrates according to the present invention at 24 hours post-injury PTBI samples.

FIG. 4A shows that ETC complex assays were measured using substrates according to the present invention in rat frozen platelets rich plasma (PRP) biofluid collected after 24 h post-injury in both Sham and PTBI. FIG. 4B shows that ETC Complexes (Complex I, II, and IV) were measured using substrates according to the present invention in swine frozen PRP biofluid samples collected at pre-injury condition and 4 h post-injury.

FIGS. 5A-5C is a series of graphs showing mitochondrial dysfunction (enzyme complex activities) measured as oxygen consumption rate in frozen rat brain mitochondrial samples following a penetrating TBI. FIG. 5A shows data for Complex I. FIG. 5B shows data for Complex II. FIG. 5C shows data for Complex IV. FIG. 5D shows data for Complex activities I, II, and IV for frozen swine brain mitochondrial samples from ipsilateral (IPSI) or injured hemisphere and contralateral (Contra) or non-injured hemisphere.

FIGS. 6A-6D is a series of graphs showing mitochondrial dysfunction (enzyme complex activities) measured as oxygen consumption rate at acute end points (FIG. 6A at 30 min.; FIG. 6B at 3 hours; FIG. 6C at 6 hours; and FIG. 6D at 24 hours) in frozen rat buffy coat samples following penetrating TBI. FIGS. 6A′-6D′ is a series of graphs showing mitochondrial dysfunction (enzyme complex activities) measured as oxygen consumption rate at acute end points for the same times, but with the Controlled Cortical Impact (CCI) injury model of TBI.

FIGS. 7A-7C is a series of graphs showing mitochondrial dysfunction (enzyme complex activities) measured as oxygen consumption rate at chronic end points (FIG. 7A at 3 days; FIG. 7B at 7 days; FIG. 7C at 14 days) in frozen rat buffy coat samples following penetrating TBI. FIG. 7D is a table showing sample size information. FIGS. 7A′-7C′ is a series of graphs showing mitochondrial dysfunction (enzyme complex activities) measured as oxygen consumption rate at chronic end points for the same times, but with the Controlled Cortical Impact (CCI) injury model of TBI. FIG. 7D′ is a table showing sample size information.

FIGS. 8A-8D is a series of graphs showing mitochondrial dysfunction (Complex enzyme activities) measured as oxygen consumption rate at acute end points (FIG. 8A at 30 min.; FIG. 8B at 3 hours; FIG. 8C at 6 hours; and FIG. 8D at 24 hours) in frozen rat platelet rich plasma (PRP) samples following penetrating TBI. FIGS. 8A′-8D′ is a series of graphs showing mitochondrial dysfunction (enzyme complex activities) measured as oxygen consumption rate at acute end points for the same times, but with the Controlled Cortical Impact (CCI) injury model of TBI.

FIGS. 9A-9B is a series of graphs showing mitochondrial dysfunction (Complex enzyme activities) measured as oxygen consumption rate (OCR) after 24 hours in fresh (FIG. 9A) and frozen (FIG. 9B) human peripheral blood mononuclear cell samples follow a hard-hit during parachute jump training. FIG. 9A illustrates the measurement of the basal rate driven by glucose, as well as the State III (combined activities of Complex I and Complex II in presence of ADP), State IV, and State V of mitochondrial respiration in fresh samples. In contrast, FIG. 9B displays the measurements of Complex I, Complex II, Antimycin A (AA) driven inhibition of Complex III, and Complex IV activities in frozen samples.

### DETAILED DESCRIPTION OF INVENTION

The present invention is directed to identification and characterization of mitochondria bioenergetics in a frozen conditions of collected tissue or biofluid samples. The analysis of mitochondrial bioenergetics may serve as qualitative and/or quantitative biomarker for validating a health condition or pathology, for example, traumatic brain injury (TBI), or evaluate treatment efficacy under, for example, TBI and/or polytrauma conditions.

In this detailed description, references to “one embodiment”, “an embodiment”, or “in embodiments” mean that the feature being referred to is included in at least one embodiment of the invention. Moreover, separate references to “one embodiment”, “an embodiment”, or “embodiments” do not necessarily refer to the same embodiment; however, neither are such embodiments mutually exclusive, unless so stated, and except as will be readily apparent to those skilled in the art. Thus, the invention can include any variety of combinations and/or integrations of the embodiments described herein.

As used herein “substantially”, “generally”, “about”, and other words of degree are relative modifiers intended to indicate permissible variation from the characteristic so modified (e.g., ±0.1%, ±0.5%, ±1.0%, ±2%, ±5%, ±10%, ±20%). It is not intended to be limited to the absolute value or characteristic which it modifies but rather possessing more of the physical or functional characteristic than its opposite, and preferably, approaching or approximating such a physical or functional characteristic.

The present invention is directed to identification and/or qualitative characterization of mitochondria bioenergetics functions in a frozen tissue or frozen biofluid sample following varying severities of TBI, including penetrating TBI and Controlled cortical impact model of TBI. In embodiments, the biofluid may comprise at least one of blood cells, platelets (e.g., plasma rich platelets (PRP)), or buffy coat samples (containing white blood cells (WBCs)).

Frozen samples are comprised of tissue or biofluids that have been preserved at freezing temperatures. In laboratory practice, the standard freezing temperature for sample preservation is −80° C. In embodiments, the frozen sample according to the present invention may have been frozen for a period of time of about 15 minutes, about 1 days, about 2 days, about 3 days, about 7 days, about 14 days, up to about a month or more.

In specific embodiments, the mitochondrial oxidative phosphorylation, OXPHOS, (e.g., ATP synthesis using ADP plus other Complex I-IV substrates) or respiration (oxygen consumption using Complex I-IV substrates without ADP) include, but are not limited to, electron transport chain (ETC) complexes, for example, at least one of ETC Complex I, ETC Complex II, or ETC Complex IV.

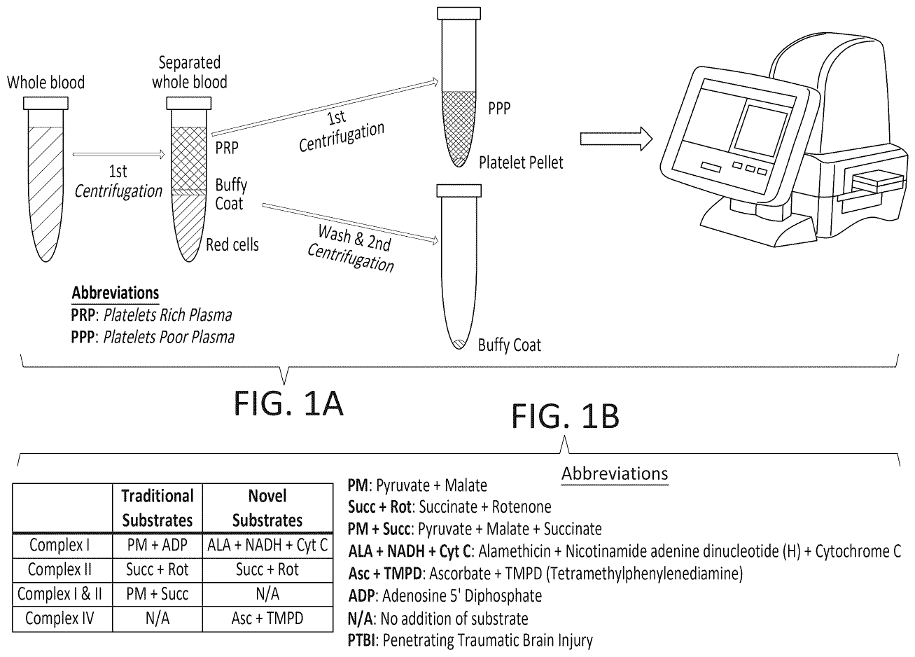

In various embodiments, the frozen sample may be measured using a commercially-available substate which may be at least one of glucose, alamethicin, nicotinamide adenine dinucleotide (H) and Cytochrome C; succinate and rotenone; Antimycin A; ascorbate and TMPD; Sodium Azide, or any combination thereof, for example, listed in the table of FIG. 1B. Succinate and rotenone are utilized to explore mitochondrial Complex II enzyme activity. Among the different complexes of the electron transport chain (ETC), complex II, also known as succinate dehydrogenase, is directly stimulated by succinate. Rotenone inhibits Complex I enzyme, thereby enabling only forward electron transfer from Complex II to Complex III.

In specific embodiments, measurement of the mitochondria bioenergetics may be measured using oxygen consumption rate (OCR) using, for example, using a biochemical assay, a spectrofluorometer, or a Seahorse XFe Analyzer. This technique measures the OCR rate at which mitochondria/tissue samples consume oxygen and produce water, which can be used to calculate their metabolic rate in ex vivo. The oxygen consumption rate (OCR) may be measured, which is an indirect measurement of the cellular energy in the form of OXPHOS or ATP (adenosine 5′ triphosphate) synthesis of mitochondria. Each assay is affected by various parameters and conditions, including, for example, at least one of the types of tissue, sample concentration/volume ratio, temperature, or the like. Consequently, the respirometry protocol should be optimized. Further, the selection of substrates determines the specific enzyme complex targeted in the ETC, thereby yielding the desired results.

In specific embodiments, an Agilent Seahorse XFe96 Seahorse Analyzer may be used. The analytical cartridge of this device comprises a two layered plate. The top of cartridge has different injection ports to inject substrates in the preloaded mitochondrial samples to measure bioenergetics in the second or lower cartridge. There are total 4 ports and each port is preloaded with different combination of substrate concentrations which can be injected in a sequential manner as set standard operating procedures in computer program. The oxygen consumption rate (OCR) is measured immediately following each substrate injection and before adding the next substrate. Each OCR value represents the respiration with a particular substrate.

Analysis and detection of mitochondrial bioenergetics in the frozen sample may allow identification of a health condition in a subject from which the frozen sample was taken. Currently, there is limitation of practical applicability of transporting the fresh tissue for the real time respirometry. However, there is no limitation of transporting the frozen tissue or biofluids for this assessment.

In neurological diseases, the OCR in brain tissue is impacted by high energy demands of neurons. A decrease in OCR is observed in conditions like stroke, TBI, and neurodegenerative diseases (e.g., Alzheimer's), indicating impaired brain function due to disrupted energy metabolism. Further, numerous pathologies result in multiple-organ failure, which is thought to be a direct consequence of compromised cellular bioenergetic status. The present invention allows the measurement of mitochondrial abnormality in frozen stored samples, which can be applicable as a potential diagnostic and theragnostic technique.

In embodiments, the health condition may include, but is not limited to, traumatic brain injury (TBI). The TBI may be mild, moderate or severe, acute or chronic, a penetrating traumatic brain injury (PTBI), a Controlled Cortical Impact (CCI) injury, a blast-induced traumatic brain injury (bTBI), or polytrauma. The health condition may be for contact, concussed, blast, stroke, and sport-related mild to severe TBI and TBI associated neurodegenerative disorders.

In an embodiment, the mitochondrial bioenergetic measurements of frozen biofluids collected from military cohorts exposed to blast or contact TBI may offer insights into individual's health status and return to duty perspective.

FIG. 1A illustrates a workflow diagram of blood processing to isolate the different cell types (e.g., buffy coat, platelets or platelets rich plasma) to measure the respiration under different conditions using Seahorse Flux Analyzer. FIG. 1B is a table that shows different sets (combination) of traditional substrates and substrates of the present invention used to measure individual ETC complexes activity under fresh and frozen tissue and mitochondrial isolation conditions. Common abbreviations used for substrates are listed.

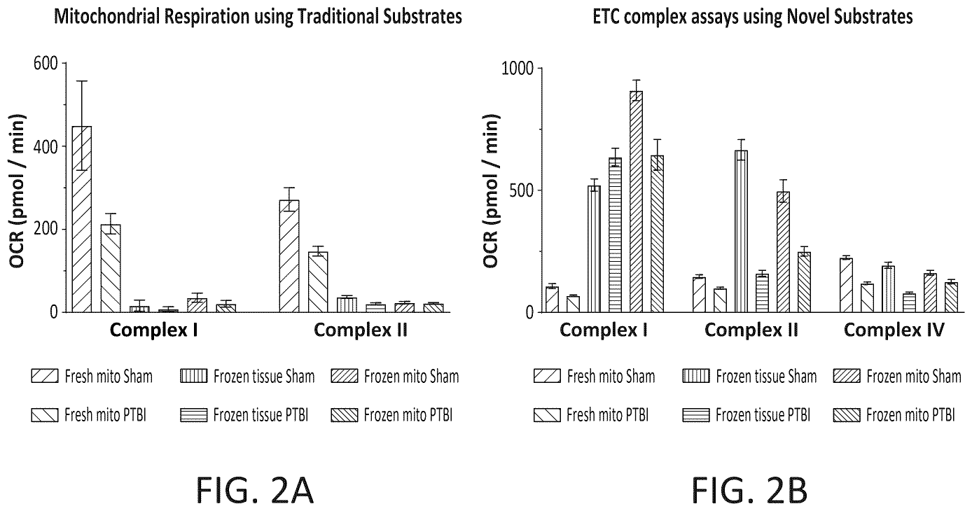

FIGS. 2A-2B show brain mitochondrial respiration measurement at 24 h post-injury in rat following penetrating TBI (PTBI). Respiration assessments (Complex I, Complex II and/or Complex IV) activity is measured in fresh and frozen both tissue and mitochondrial samples. FIG. 2A shows measured mitochondrial respiration (Complex I and Complex II) using traditional substrates. FIG. 2B shows measured ETC Complex I, II and complex IV activity using substrates of the present invention. Mitochondria: Fresh mitochondria isolated immediately after tissue isolation at 24 hours post-injury. Frozen tissues such as tissue homogenates were frozen immediately in liquid nitrogen (−80° C.) for 15 mins and then thawed at room temperature and processed for mitochondrial isolation and respiration measurements. For frozen mitochondria, isolated mitochondrial samples were frozen at −80° C. for 15 mins after isolation and protein estimation.

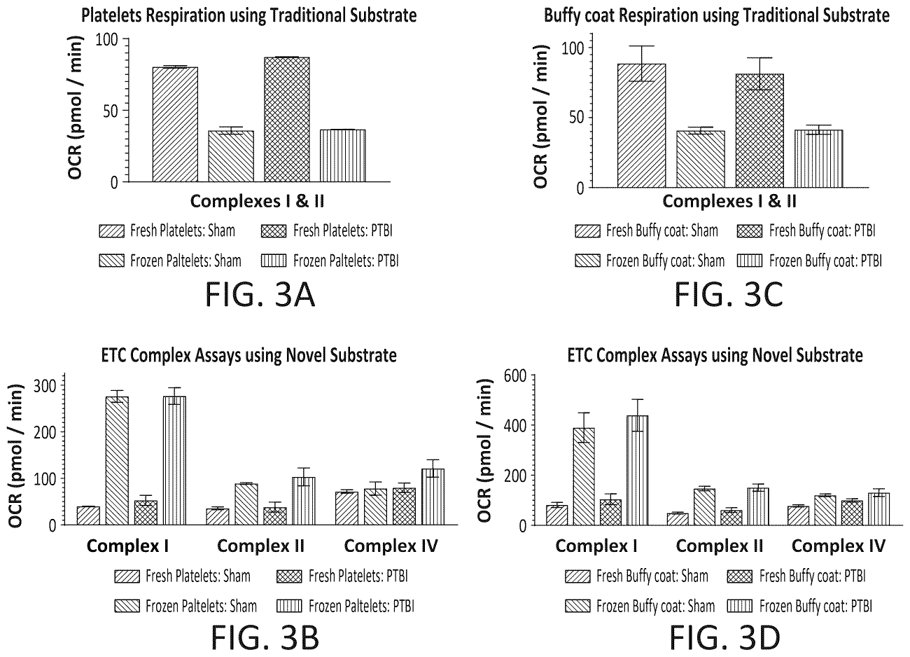

FIGS. 3A-3D are graphs that represent biofluids (platelets and buffy coat) mitochondrial respiration measurements at 24 h post-injury in rats following PTBI. As shown in the legend, FIGS. 3A-3B) showed platelet samples mitochondrial respiration using traditional substrates and substrates of the present invention at 24 hours post-injury PTBI samples. FIGS. 3C-3D show buffy coat sample mitochondrial respiration using traditional substrates and substrates of the present invention at 24 hours post-injury PTBI samples. Fresh platelets and buffy coat samples were isolated from fresh blood and measured respiration immediately. For frozen platelets, samples were frozen at −80° C. for 15 mins following isolation and protein estimation. For frozen buffy coat, samples were frozen at −80° C. for 15 mins following isolation and protein estimation.

Overall, data in the FIGS. 3A and 3C indicated that the traditional substrates set is unable to measure frozen platelet and buffy coat respiration in both PTBI and Sham condition compared to fresh platelet and buffy coat counterparts. Moreover, FIGS. 3B and 3D demonstrated successful evaluation of Complexes I, II and IV activities from frozen platelets and buffy coats at 24 hours post-injury after PTBI in rats.

When the substrates of the present invention for platelets respiration were utilized in FIG. 3B, frozen platelets exhibited mitochondrial ETC complex I, II, and IV enzyme activities that reflected changes in the respiration functions in PTBI vs Sham. Specifically, Complex IV activity showed an increasing trend in frozen platelets following PTBI vs. Sham after 24 h post-injury. FIG. 3D indicated successful evaluation of frozen buffy coat mitochondrial ETC complex activities after PTBI. More specifically, frozen buffy coat enables mitochondrial ETC complex I, II, and IV enzyme activities following PTBI vs Sham. The ETC complexes I, II, and IV activities showed identical trends in frozen buffy coat between PTBI vs. Sham at 24 hours post-injury.

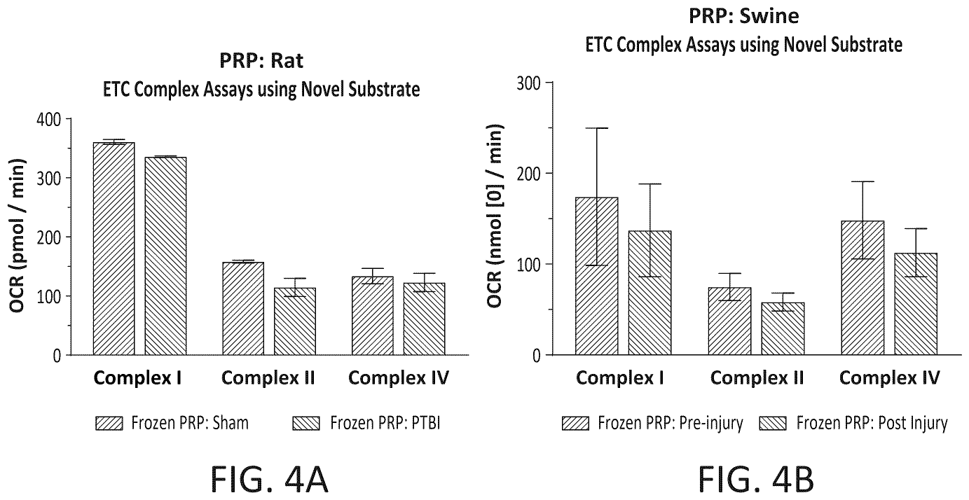

To relate the translational capability of biofluids bioenergetics function, mitochondrial complex activities in both rodent and swine species after PTBI were measured. As shown in FIG. 4A, ETC complex assays were measured using substrates of the present invention in rat frozen platelets rich plasma (PRP) biofluid collected after 24 h post-injury in both Sham and PTBI. In FIG. 4B, ETC Complexes (Complex I, II, and IV) were measured from swine frozen PRP biofluid samples collected at pre-injury condition and 4 h post-injury. Note that the PRP samples were frozen at −80° C. for 15 mins following collection of fresh plasma followed by protein estimation. After thawing at room temperature, samples (pre- or post-injury) were further processed for respiration measurements using substrates of the present invention in a same analytical plate.

From FIG. 4A, data indicated that the Complexes I, II and IV activities were measurable in frozen PRP samples and were comparable between Sham vs. PTBI after 24 h post-injury in rat. Similarly, FIG. 4B data indicated that the Complexes I, II, and IV activities in frozen PRP samples isolated from swine pre-injury and 4 h post-injury conditions were measurable. More specifically, Complex I, II and IV activities showed decreasing trend of respiratory function following PTBI when compared pre-vs. post-injury samples. The data illustrated an applicability of these assays for future TBI and polytrauma conditions, and to evaluate long-term changes in mitochondrial bioenergetics functions after, for example, at least one of TBI, PTBI, or polytrauma.

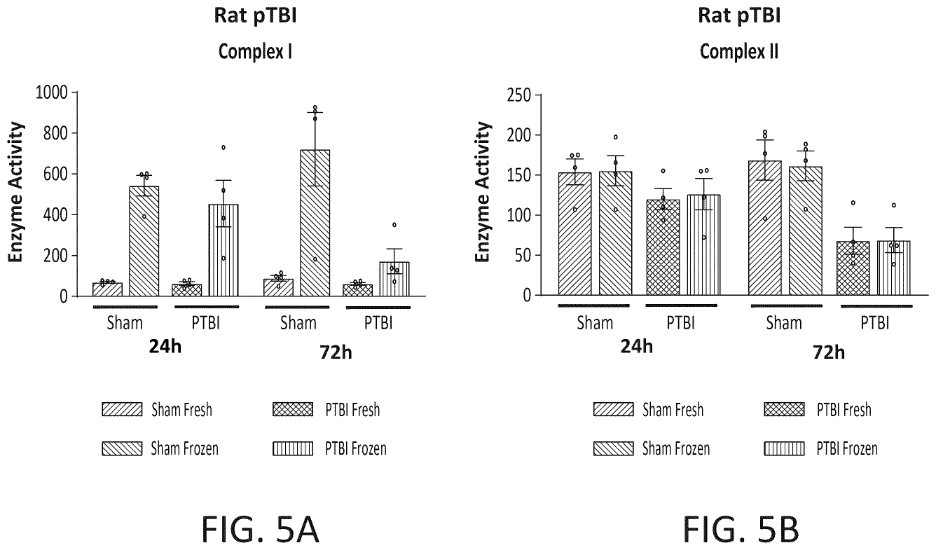

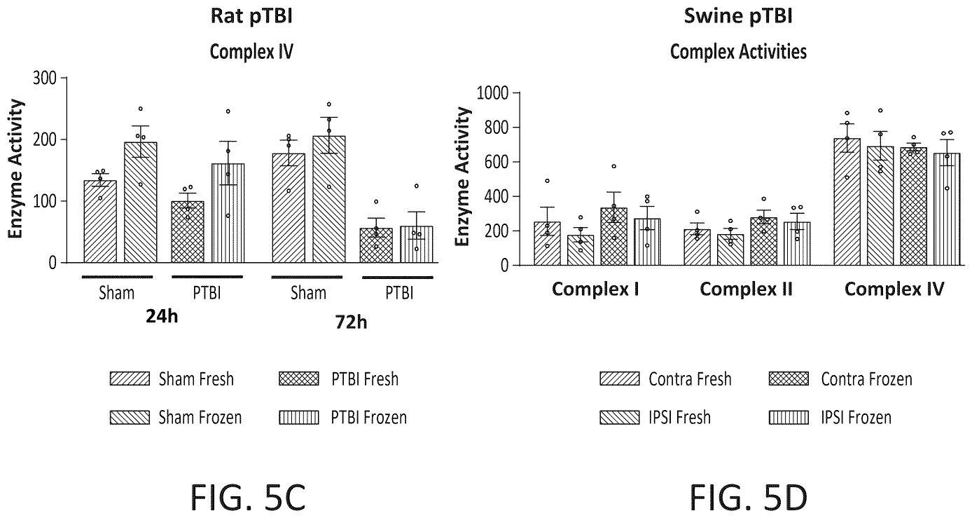

FIGS. 5A-5C is a series of graphs showing mitochondrial dysfunction (enzyme complex activities) measured as oxygen consumption rate in frozen rat brain mitochondrial samples following penetrating TBI using the substrates according to the present invention. FIG. 5A shows data for Complex I. FIG. 5B shows data for Complex II. FIG. 5C shows data for Complex IV. FIG. 5D shows data for Complex Activities I, II, and IV for frozen swine brain mitochondrial samples using the substrates according to the present invention. Thus, mitochondrial dysfunction was detected.

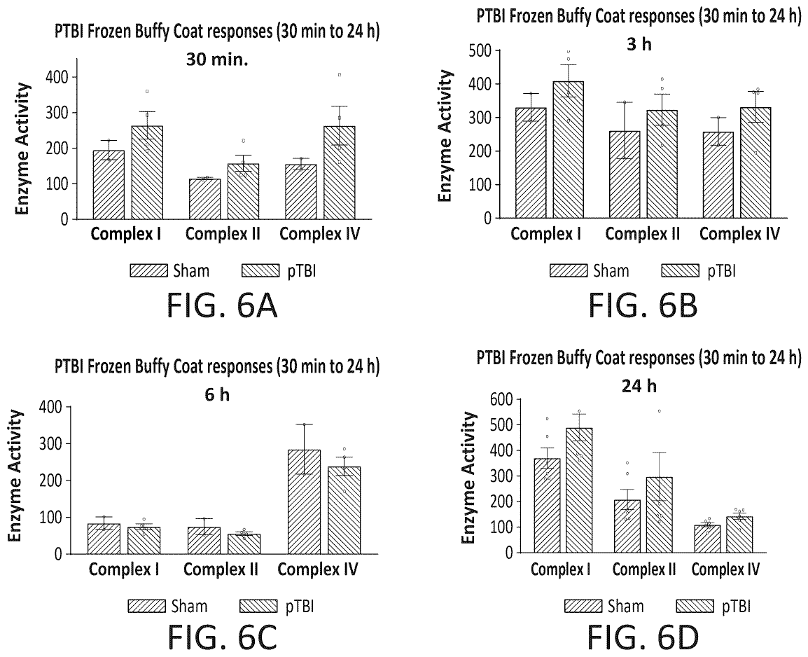

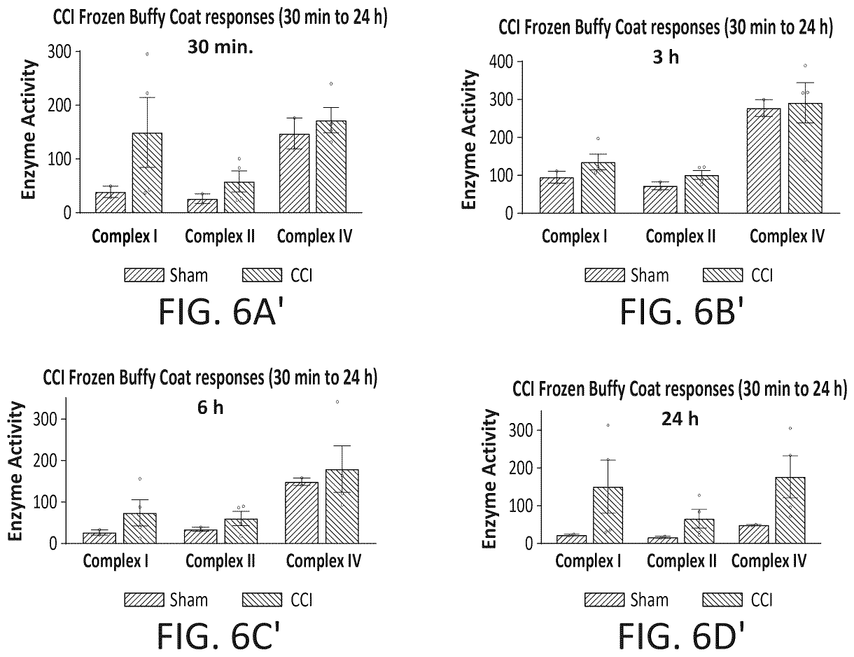

To enhance the validation of biofluids' bioenergetics function, a temporal study was conducted by pursuing an additional model of TBI (CCI) using the homologous combination of substrates, providing deeper insights into its applicability and significance. FIGS. 6A-6D is a series of graphs showing mitochondrial dysfunction (enzyme complex activities) measured as oxygen consumption rate at acute end points (FIG. 6A at 30 min.; FIG. 6B at 3 hours; FIG. 6C at 6 hours; and FIG. 6D at 24 hours) in frozen rat buffy coat samples following penetrating TBI using the substrates according to the present invention. The methods according to the present invention detected elevated responses in frozen buffy coat mitochondrial enzyme complex activities during acute post-injury time-points following mild and severe TBI. FIGS. 6A′-6D′ is a series of graphs showing mitochondrial dysfunction (enzyme complex activities) measured as oxygen consumption rate at acute end points for the same times, but validated with the Controlled Cortical Impact (CCI) model of TBI.

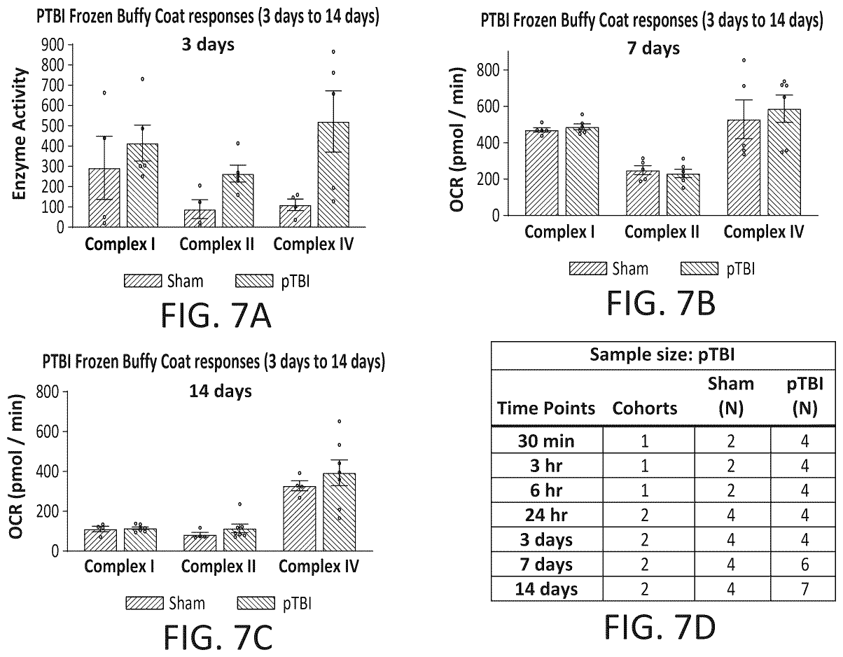

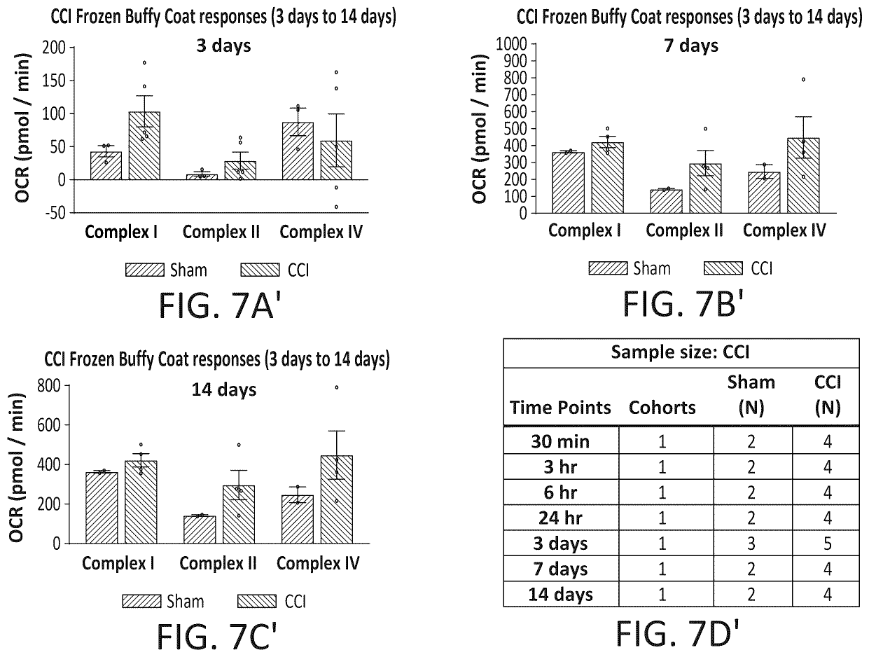

FIGS. 7A-7C is a series of graphs showing mitochondrial dysfunction (enzyme complex activities) measured as oxygen consumption rate at chronic end points (FIG. 7A at 3 days; FIG. 7B at 7 days; FIG. 7C at 14 days) in frozen rat buffy coat samples following penetrating TBI using the substrates according to the present invention. FIG. 7D is a table showing sample size information. The methods according to the present invention detected elevated responses in frozen buffy coat mitochondrial enzyme complex activities during chronic post-injury time-points following mild and severe TBI. FIGS. 7A′-7C′ is a series of graphs showing mitochondrial dysfunction (enzyme complex activities) measured as oxygen consumption rate at chronic end points for the same times, but validated with the Controlled Cortical Impact (CCI) injury model of TBI. FIG. 7D′ is a table showing sample size information.

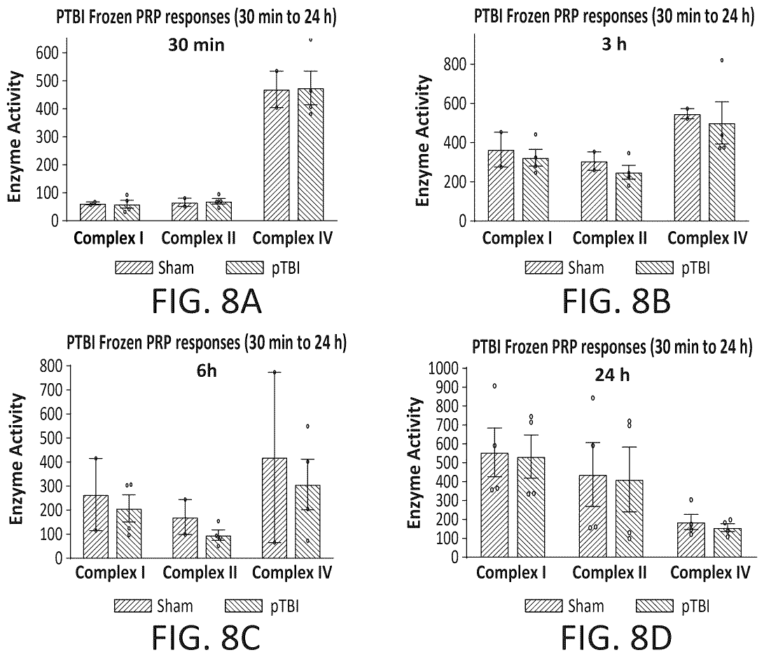

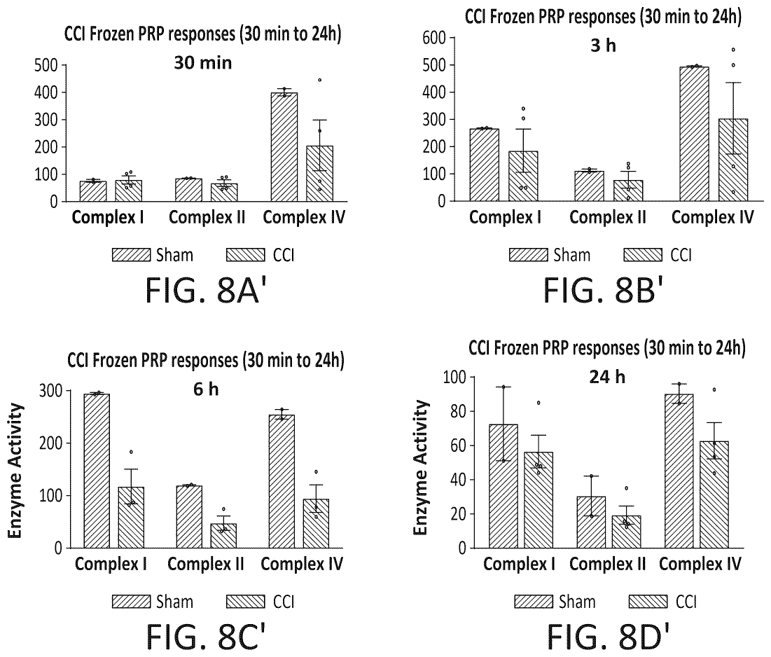

FIGS. 8A-8D is a series of graphs showing mitochondrial dysfunction (enzyme complex activities) measured as oxygen consumption rate at acute end points (FIG. 8A at 30 min.; FIG. 8B at 3 hours; FIG. 8C at 6 hours; and FIG. 8D at 24 hours) in frozen rat platelet rich plasma (PRP) samples following penetrating TBI using the substrates according to the present invention. The methods according to the present invention detected decreased responses in frozen PRP mitochondrial enzyme complex activities during acute post-injury time-points following mild and severe TBI. FIGS. 8A′-8D′ is a series of graphs showing mitochondrial dysfunction (enzyme complex activities) measured as oxygen consumption rate at acute end points for the same times, but validated with the Controlled Cortical Impact (CCI) injury model of TBI.

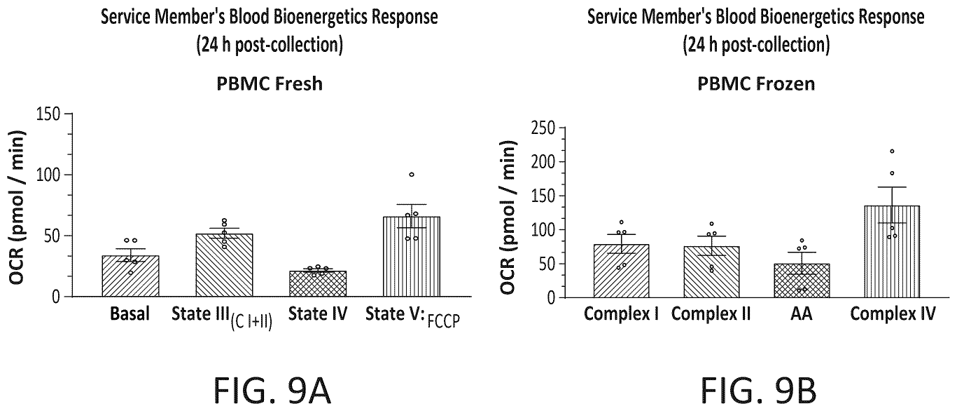

FIGS. 9A-9B is a series of graphs showing mitochondrial dysfunction (enzyme complex activities) measured as oxygen consumption rate after 24 hours in fresh (FIG. 9A) and frozen (FIG. 9B) human peripheral blood mononuclear cell samples follow a hard hit during parachute jump training. The methods according to the present invention detected mitochondrial enzyme complex activities in frozen human blood PBMC samples, thereby showing applicability in humans.

Although the present invention has been described in terms of particular exemplary and alternative embodiments, it is not limited to those embodiments. Alternative embodiments, examples, and modifications which would still be encompassed by the invention may be made by those skilled in the art, particularly in light of the foregoing teachings.

Those skilled in the art will appreciate that various adaptations and modifications of the exemplary and alternative embodiments described above can be configured without departing from the scope and spirit of the invention. Therefore, it is to be understood that, within the scope of the appended claims, the invention may be practiced other than as specifically described herein.

What is claimed is:

1. A method of assessing mitochondria bioenergetics, comprising:
obtaining a frozen tissue or frozen biofluid sample of an individual; and
measuring mitochondria bioenergetics of the frozen tissue or frozen biofluid sample using a substrate for the frozen tissue or biofluid sample, wherein the substrate comprises at least one of:
Glucose, Alamethicin, Nicotinamide adenine dinucleotide (H), and Cytochrome C;
Succinate and Rotenone; or
Ascorbate and Tetramethylphenylenediamine.

2. A method according to claim 1, wherein the measuring comprises measuring mitochondrial oxidative function by respirometry function assessment.

3. A method according to claim 1, wherein the measuring comprises measuring at least one of mitochondrial electron transport chain enzyme complex I, electron transport chain enzyme complex II, or electron transport chain enzyme complex IV activities in a frozen biofluid sample obtained after at least one of traumatic brain injury (TBI) or penetrating TBI.

4. A method according to claim 3, wherein the frozen tissue or frozen biofluid samples were collected at 30 minutes to 24 hours following mild or severe TBI.

5. A method according to claim 3, wherein the frozen tissue or frozen biofluid samples were collected between 3 days to about 14 days following mild or severe TBI.

6. A method according to claim 3, wherein a frozen biofluid sample comprises buffy coat layers.

7. A method according to claim 3, wherein a frozen biofluid sample comprises platelet rich plasma.

8. A method according to claim 3, wherein a frozen biofluid sample comprises blood platelets.

9. A method according to claim 1, where the frozen tissue or frozen biofluid sample is from a human having traumatic brain injury.

10. A method according to claim 1, further comprising identifying a health status or condition of the individual based on said measuring.

11. A method according to claim 1, further comprising diagnosing traumatic brain injury or polytrauma based on said measuring.

12. A method according to claim 1, further comprising diagnosing diabetes or ischemic injury based on said measuring.

13. A method according to claim 1, further comprising diagnosing a neurodegenerative condition or disease based on said measuring.

14. A method according to claim 11, wherein the neurodegenerative condition or disease comprises Alzheimer's disease.

15. A method according to claim 1, wherein the frozen tissue or frozen biofluid sample is thawed before said measuring.
